(19) United States
(12) Patent Application Publication
(10) Pub. No.: US 2009/0187066 A1
(43) Pub. Date: Jul. 23, 2009

(54) APPARATUS AND METHOD FOR TREATING URINARY INCONTINENCE

(75) Inventors: Firouz Daneshgari, Akron, OH (US); Adonis Hijaz, Manlius, NY (US)

Correspondence Address: TAROLLI, SUNDHEIM, COVELL & TUMMINO L.L.P., 1300 EAST NINTH STREET, SUITE 1700, CLEVEVLAND, OH, 44114, US

(73) Assignee: The Cleveland Clinic Foundation

(21) Appl. No.: 12/212,693

(22) Filed: Sep. 18, 2008

Related U.S. Application Data
(60) Provisional application No. 60/973,409, filed on Sep. 18, 2007.

(51) Int. Cl. A61F 2/00

(52) U.S. Cl. 600/30; 600/37

(57) ABSTRACT

An apparatus and method for treating urinary incontinence in a female subject includes an elongated urethral sling having a central support portion intermediate first and second end portions. The central support portion includes first and second ends. The first and second end portions are respectively coupled to and extend from the first and second ends of the central support portion. The central support portion is made of a non-resorbable polymer mesh, and the first and second end portions are made of a polymer mesh different than the non-resorbable polymer mesh.

### RELATED APPLICATION

This application claims priority from U.S. provisional patent application Ser. No. 60/973,409, filed Sep. 18, 2008, the entirety of which is hereby incorporated by reference.

### TECHNICAL FIELD

The present invention relates generally to an apparatus and method for treating genitourinary disorders, and more particularly to an elongated urethral sling and method for treating urinary incontinence in female subjects.

### BACKGROUND OF THE INVENTION

Urinary incontinence is a disorder that generally affects women of all ages. The inability to control urination can impact a subject both physiologically and psychologically. Urinary incontinence can interfere with a subject's daily activity and impair quality of life. Stress urinary incontinence is one type of urinary incontinence. Actions including straining, coughing, and heavy lifting can cause women with stress urinary incontinence to void urine involuntarily.

Various physiological conditions cause urinary incontinence in women. Stress urinary incontinence is generally caused by two conditions that occur independently or in combination. One condition, known as intrinsic sphincter deficiency (ISD), occurs when the urethral sphincter fails to coapt properly. ISD may cause urine to leak out of the urethra during stressful actions. A second condition, known as hypermobility, occurs when the pelvic floor is weakened or damaged and causes the bladder neck and proximal urethra to rotate and descend in response to increases in intra-abdominal pressure. When intra-abdominal pressure increases due to strain resulting from coughing, for example, urine leakage often results.

One method for treating stress urinary incontinence includes placing a sling to either compress the urethral sphincter or placing a sling to provide a “back stop” to the bladder neck and proximal urethra. Providing support to the bladder neck and proximal urethra maintains the urethra in the normal anatomical position, while elevation places the urethra above the normal anatomical position. Current sling-based methods for treating urinary incontinence typically require dissection of the anterior vaginal wall, which can result in irreparable nerve damage and scarring.

### SUMMARY OF THE INVENTION

In one aspect of the present invention, an apparatus for treating urinary incontinence in a female subject comprises an elongated urethral sling having a central support portion intermediate first and second end portions. The central support portion includes first and second ends. The first and second end portions are respectively coupled to and extend from the first and second ends of the central support portion. The central support portion is comprised of a non-resorbable polymer mesh, and the first and second end portions are comprised of a polymer mesh different than the non-resorbable polymer mesh.

In another aspect of the present invention, a method is provided for treating urinary incontinence in a female subject. One step of the method includes providing an elongated urethral sling having a central support portion intermediate first and second end portions. The central support portion includes first and second ends. The first and second end portions are respectively coupled to and extend from the first and second ends of the central support portion. The central support portion is comprised of a non-resorbable polymer mesh, and the first and second end portions are comprised of a polymer mesh different than the non-resorbable polymer mesh. The urethral sling is implanted in the subject so that the central support portion is taught against the anterior vaginal wall and provides support to the urethra.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 is a perspective view showing an apparatus for treating urinary incontinence in a female subject constructed in accordance with the present invention;

FIG. 2A is an exploded perspective view of the apparatus in FIG. 1 shows first and second end portions and a central support portion;

FIG. 26 is a cross-sectional view of the central support portion taken along Line 2B-2B in FIG. 2A;

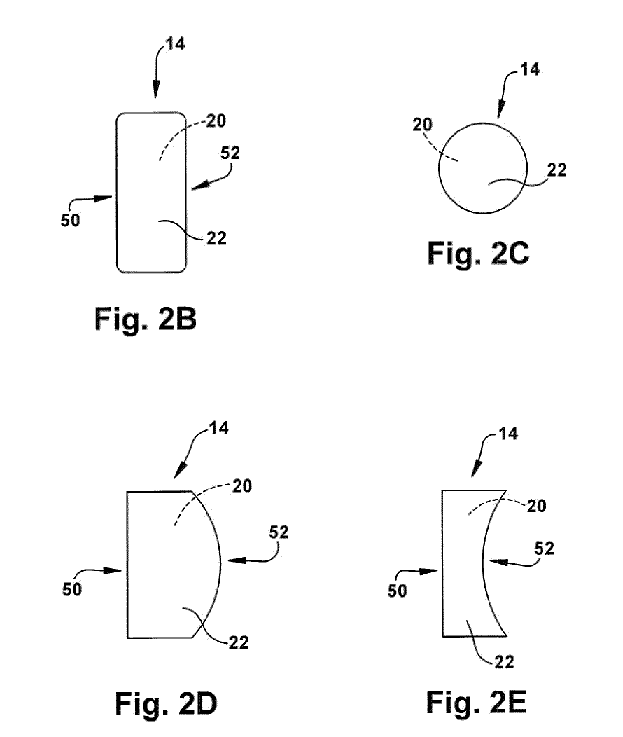

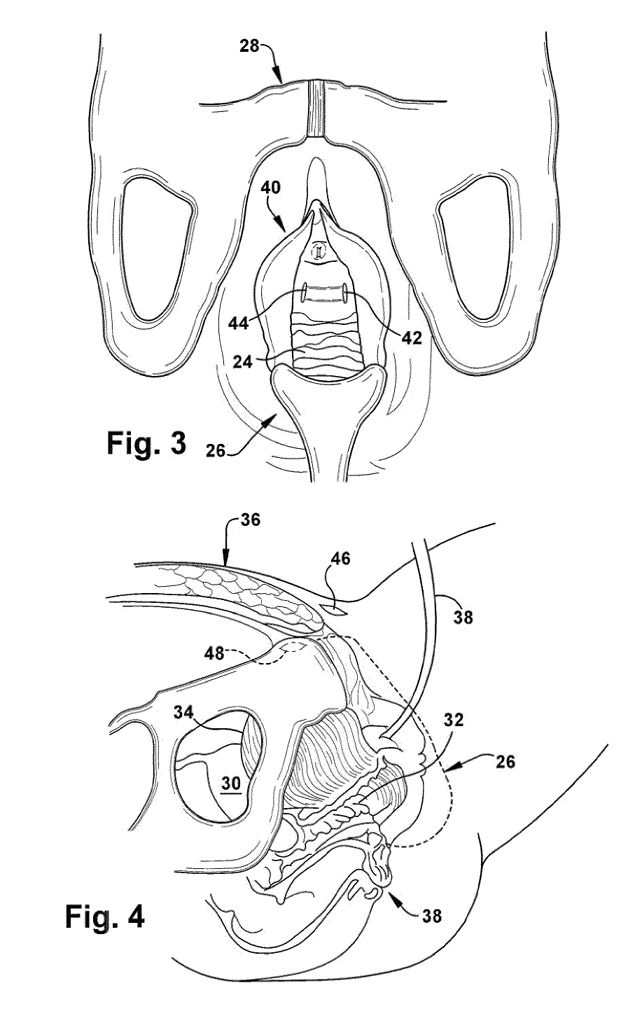

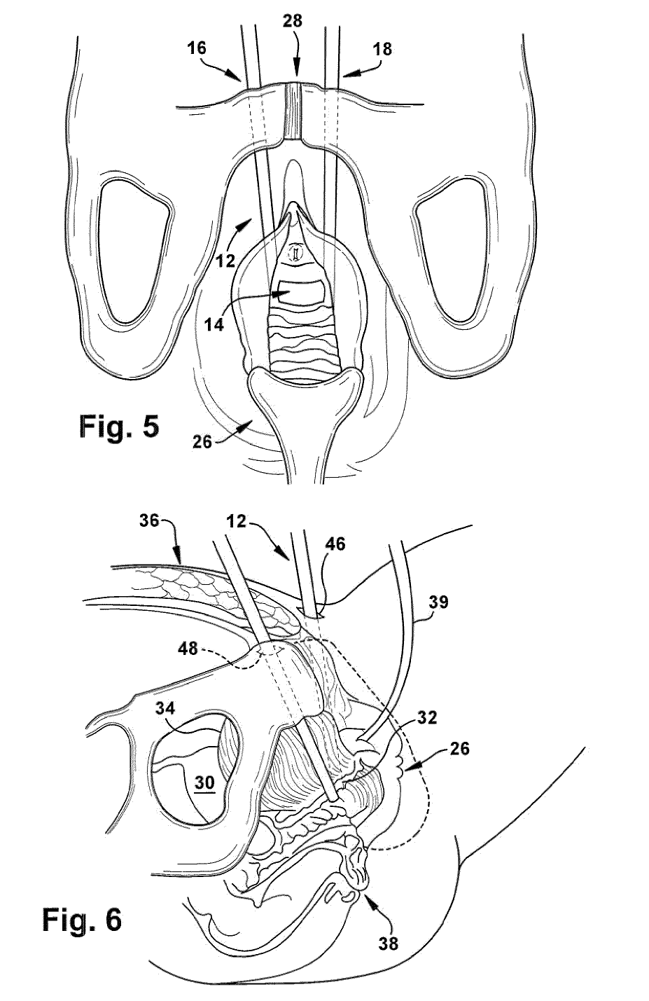

FIG. 2C is a cross-sectional view showing an alternative embodiment of the central support portion in FIG. 2B;

FIG. 2D is a cross-sectional view showing another alternative embodiment of the central support portion in FIG. 2B;

FIG. 2E is a cross-sectional view showing an alternative embodiment of the central support portion in FIG. 2B;

FIG. 3 is a perspective view showing two lateral stab incisions through the anterior vaginal wall;

FIG. 4 is a sagittal section of a female pelvis showing two lateral stab incisions at the suprapubic region of the pelvis;

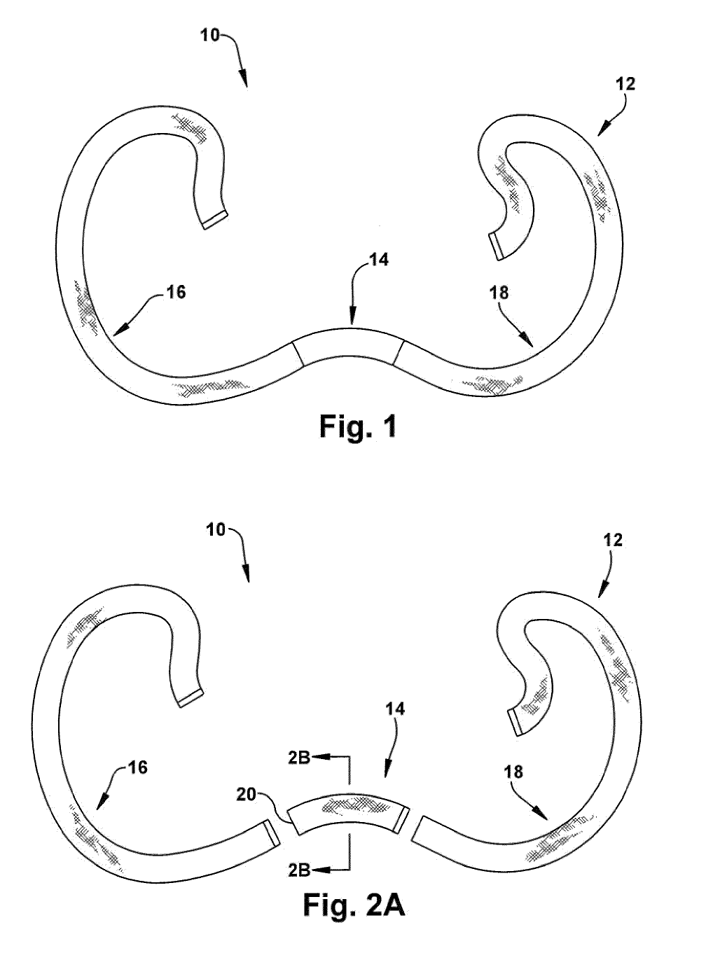

FIG. 5 is a perspective view showing a central support portion of the apparatus in FIG. 1 pulled taught against the anterior vaginal wall; and

FIG. 6 shows the sagittal section of FIG. 4 with the apparatus of FIG. 1 implanted in a subject.

### DETAILED DESCRIPTION

The present invention relates generally to an apparatus and method for treating genitourinary disorders, and more particularly to an elongated urethral sling and method for treating urinary incontinence in female subjects. As representative of the present invention, FIGS. 1 and 2 illustrate an apparatus 10 for treating urinary incontinence in a female subject. Prior art methods for treating urinary incontinence using urethral slings typically include anterior vaginal wall dissection and sub-urethral sling placement. Nerve terminals are distributed in the proximal and middle portion of the urethra, making them vulnerable to injuries occurring during vaginal dissection (e.g., incisions made on the anterior vaginal wall at the middle of the urethra). Unlike the prior art, the present invention provides a nerve-sparing urethral sling and related method for treating urinary incontinence that does not require dissection of the anterior vaginal wall and, thus, mitigates the risk of extensive nerve damage to the nerve terminals distributed in the proximal and middle portion of the urethra.

Unless otherwise defined, all technical terms used herein have the same meaning as commonly understood by one of ordinary skill in the art to which the present invention pertains.

In the context of the present invention, the term “urinary incontinence” can refer to disorders in which subjects lack voluntary control of normal urinary function. One form of urinary incontinence includes overflow incontinence, in which contractile dysfunction of the detrusor muscle results in large bladder volumes and urinary dribbling. Another form of urinary incontinence includes stress incontinence, in which subjects lose small volumes of urine as a result of temporarily increased abdominal pressure. Additionally, urinary incontinence includes functional incontinence, in which normally continent subjects are incontinent as a result of physical or cognitive problems, or various medications, such diuretics.

As used herein, the term “subject” can refer to any warm-blooded organism including, but not limited to, human beings, pigs, rats, mice, dogs, goats, sheep, horses, monkeys, apes, rabbits, cattle, etc.

As used herein, the term “polymer mesh” can refer to an interwoven or intertwined polymer structure or network. A polymer mesh can formed from any knit, woven, or knotted polymer or combination of polymers of open texture. The term can also refer to any arrangement of interlocking polymers with spaced apart, substantially uniform small openings between the polymers.

As used herein, the term “non-resorbable polymer” can refer to a polymer that exhibits relatively limited or no in vivo degradation.

As shown in FIGS. 1 and 2A-E, one embodiment of the present invention includes an apparatus 10 comprising an elongated urethral sling 12. The urethral sling 12 has a generally flattened, rectangular (or substantially rectangular) configuration. Depending upon the intended anatomical placement and the particular type of urinary incontinence affecting a subject, the shape of the urethral sling 12 may be varied as needed. For example, the urethral sling 12 may have a trapezoidal shape, a hexagonal shape, or an elliptical shape. It will be appreciated that the dimensions of the urethral sling 12 (i.e., the length, width and thickness) may also be varied depending upon the anatomical placement of the urethral sling and the particular type of urinary incontinence affecting the subject.

The urethral sling 12 includes a central support portion 14 intermediate first and second end portions 16 and 18. The central support portion 14 is adapted for placement against the anterior vaginal wall 24 (FIG. 3) of a subject, and includes oppositely disposed first and second ends 20 and 22 (FIG. 2A). As shown in FIG. 2B, the central support portion 14 also includes oppositely disposed first and second major surfaces 50 and 52 that are substantially parallel to one another. To facilitate placement of the central support portion 14 against the anterior vaginal wall 24 (FIG. 3), the first and/or second major surfaces 50 and 52 (FIG. 2B) can have a shape that mirrors the shape of the anterior vaginal wall.

Although the central support portion 14 is shown in FIGS. 2A-B as having a substantially rectangular shape, it will be appreciated that the central support portion can have other configurations as well. For example, the central support portion 14 can have a trapezoidal, hexagonal, elliptical or square-like shape. The length, width, and thickness of the central support portion 14 can be varied as needed. For example, the central support portion can have a length of about 4 inches, a width of about 2 inches, and a thickness of about 0.01 inches to about 0.5 inches.

To facilitate placement of the central support member 14 against the anterior vaginal wall 24 (FIG. 3), the cross-sectional shape of the central support portion (FIG. 2C) can be varied as needed. As shown in FIG. 2C, for example, the central support portion 14 can have a non-flattened (e.g., tubular) configuration. Alternatively, the second major surface 52 of the central support portion 14 can have a convex (FIG. 2D) or concave (FIG. 2E) shape with respect to the first major surface 50. It should be appreciated that the first and/or second major surfaces 50 and 52 can also have an irregular shape to facilitate placement of the central support portion 14 against the anterior vaginal wall 24 (FIG. 3).

The central support portion 14 (FIGS. 1 and 2A-E) is made of a synthetic, non-resorbable polymer mesh, such as polypropylene. The non-resorbable polymer mesh may have any desired configuration, including any number of knits, weaves, braids, or non-wovens, such as those described in U.S. Pat. Nos. 5,569,273, 5,124,136, 5,292,328, 5,002,551, 4,838,884, 4,655,221, 4,652,264, and 4,633,873, the entireties of which are hereby incorporated by reference. The construction of the central support portion 14 can be varied to impart the central support portion with a desired flexibility. For example, the central support portion 14 can have a dense weave or knit construction to impart the central support portion with less flexibility. The construction of the central support portion 14 can also be varied to impart different functional properties to the central support portion. For example, the central support portion 14 can have a knit construction to allow tissue in-growth. Additionally or alternatively, the central support portion 14 can be constructed to give the central support portion a pre-formed, curvilinear shape that allows the central support portion to more readily conform to the shape of anterior vaginal wall 24 (FIG. 3).

The non-resorbable polymer mesh used to form the central support portion 14 (FIG. 1) may be single-ply, double-ply, or greater. The central support portion 14 can have a homogenous composition, i.e., the central support portion can be formed from a single type of non-resorbable polymer mesh. Alternatively, the central support portion 14 can have a heterogeneous configuration. For example, the central support portion 14 can comprise a core made of a first non-resorbable polymer mesh and an outer layer surrounding the core that is made from a second non-resorbable polymer mesh different than the first non-resorbable polymer mesh. It will be appreciated that the central support portion 14 can also be entirely or partly made of a solid material (e.g., a hardened plastic).

The non-resorbable polymer mesh used to form the central support portion 14 can include any one or combination of known synthetic polymers. Examples of synthetic non-resorbable polymers that may be used to form the central support portion 14 can include, but are not limited to, nylon, polyethylene, polyester, polypropylene, fluoropolymers, such as polytetrafluoroethylene (PTFE) and expanded PTFE (ePTFE), fluorinated ethylene propylene, DACRON, and copolymers thereof. Other examples of synthetic non-resorbable polymers that may be used to form the central support portion 14 are known in the art.

The first and second end portions 16 and 18 of the urethral sling 12 have an elongated, substantially rectangular shape. As shown in FIG. 1, the shape (i.e., length, width and thickness) of the first and second end portions 16 and 18 can be identical to the shape of the central support portion 14. For example, the length of each of the first and second end portions 16 and 18 can be about 15 inches, the width of each of the first and second end portions can be about 2 inches, and the thickness of each of the first and second end portions can be about 0.01 inches to about 0.5 inches. It will be appreciated, however, that the shape of the first and second end portions 16 and 18 can be different than the shape of the central support portion 14.

The first and second end portions 16 and 18 are made of a polymer mesh, such as a synthetic or non-synthetic polymer mesh that is different from the non-resorbable polymer mesh of the central support portion 14. For example, the central support portion 14 can be comprised of a polypropylene mesh and each of the first and second end portions 16 and 18 can be comprised of PTFE or ePTFE. Examples of other polymer meshes that may be used to form the first and second end portions 16 and 18 can include, but are not limited to, nylon, polyethylene, polyester, polypropylene, fluorinated ethylene propylene, DACRON, and copolymers thereof. Additional examples of polymers that may be used to form the first and second end portions 16 and 18 are known in the art.

The polymer mesh used to form the first and second end portions 16 and 18 may have any of a number of knits, weaves, braids or non-wovens, such as those provided above. The construction of the first and second end portions 16 and 18 can be varied to impart the first and second end portions with a desired flexibility. For example, the first and second end portions 16 and 18 can have a dense weave or knit construction to impart the first and second end portions with less flexibility. It will be appreciated that the flexibility of the first and second end portions 16 and 18 can be the same or different. Additionally, it will be appreciated that the flexibility of the first and second end portions 16 and 18 can be the same as, or different from, the flexibility of the central support portion 14.

The polymer mesh used to form the first and second end portions 16 and 18 may be single-ply, double-ply, or greater. Each of the first and second end portions 16 and 18 can have a homogenous composition, i.e., each of the first and second end portions can be formed from a single type of polymer mesh. Alternatively, each of the first and second end portions 16 and 18 can have a heterogeneous configuration. For example, each of the first and second end portions 16 and 18 can comprise a core made of a first polymer mesh and an outer layer surrounding the core that is made from a second different polymer mesh. It will be appreciated that the first and second end portions 16 and 18 can be entirely or partly made of a solid material (e.g., a hardened plastic). It will also be appreciated that the homogeneity or heterogeneity of the first and second end portions 16 and 18 can be the same or different, and that the homogeneity or heterogeneity of the end portions can be the same as, or different from, the central support portion 14.

Referring to FIG. 1, the first and second end portions 16 and 18 of the urethral sling 12 are respectively coupled to and extend from the first and second ends 22 and 24 of the central support portion 14. The first and second ends 22 and 24 can be coupled to the first and second end portions 16 and 18, respectively, using any one or a variety of different coupling mechanisms (not shown). Non-limiting examples of coupling mechanisms can include staples, sutures, pins, fasteners and adhesives. For example, the first and second ends 22 and 24 can be coupled to the first and second end portions 16 and 18, respectively, using 3/0 VICRYL sutures. The coupling mechanisms allow the first and second ends 22 and 24 to be in physical contact, but not integrally formed with, the first and second end portions 16 and 18 (respectively). Alternatively, it will be appreciated that at least a portion of the first end portion 16, the second end portion 18, and the central support portion 14 can include a thermoplastic polymer so that the first and second ends 22 and 24 can be thermally bonded to the first and second end portions (respectively).

At least a portion of the urethral sling 12 may be treated with one or a combination of the same or different drugs prior to implantation to facilitate biocompatibility of the urethral sling and prevent infection. For example: only the central support portion 14 can be treated with a drug; only the first major surface 50 of the central support portion can be treated with a drug; or, alternatively, the first major surface can be treated with a first drug while the second major surface 52 can be treated with a drug different than the first drug. Exemplary drugs can include, but are not limited to, antimicrobials, such as neomycin, and anti-inflammatory agents, such as NSAIDs. The drug(s) may be released into the tissue of the subject immediately upon, or sometime after, implanting the urethral sling 12.

It will be appreciated that one or more portions of the urethral sling 12 can be treated with a hydrophobic coating to promote bonding between a drug and the urethral sling and/or to affect the release rate of the drug(s). Examples of hydrophobic coatings can include, but are not limited to, PTFE, silicon, and pyrelene. Hydrophobic coatings may be used in conjunction with hydrophobic drugs, such as ibuprofen, ketoprofen, diclofenac, and lidocaine in hydrophilic form. Where bonding between a hydrophobic coating and a drug is weak, the absorbed drug may be readily released and delivered upon contact with the subject's tissues. Alternatively, a stronger bonding affinity between the hydrophobic coating and the drug may provide a desired release rate of the drug from the urethral sling 12.

Another embodiment of the present invention includes a method for treating urinary incontinence in a female subject. The relevant parts of the female lower abdomen are disclosed in FIGS. 3-6. In FIGS. 3 and 5, the anterior vaginal wall 24, the vagina 26, and the pubic bone 28 are shown. In FIGS. 4 and 6, the vagina 26, the uterus 30, the urethra 32, the pubic bone 28, the urinary bladder 34, and the abdominal wall 36 are shown. Although the method of the present invention is exemplified below for treating a female subject suffering from stress urinary incontinence, it will be appreciated that the method of the present invention can be used to treat other forms of urinary incontinence.

As discussed above, physiological conditions that cause stress urinary incontinence include intrinsic sphincter deficiency (ISD), bladder neck hypermobility, and combinations thereof. When the urethral sling 12 is employed to treat these conditions, the urethral sling may be used to provide support to a subject's urethra 32. Where the physiological condition is ISD, for example, the urethral sling 12 may be implanted to improve improper coaption of the urethral sphincter (not shown in detail). Alternatively, where the physiological condition is hypermobility, the urethral sling 12 may be implanted to support, elevate, or “back stop” the midurethra. In a subject suffering from a combination of ISD and hypermobility, the urethral sling 12 may be implanted to support one or both of these sites.

In an example of the method, a suprapubic approach is used to treat female stress incontinence. It will be appreciated, however, that other surgical methods for implanting the urethral sling 12, such as transvaginal, transabdominal, pre-pubic, transobturator, or some combination of these approaches may also be used to implant the urethral sling. In general, the suprapubic procedure involves forming a track between abdominal incisions located behind both the pubic bone 28 and the vagina 26. The ends of a urethral sling are then drawn back up through the tracks to place a looped portion of the sling underneath the mid-urethra.

According to the superpubic approach of the present invention, a subject can first receive broad spectrum antibiotics, such as gentamicin and ampicillin to prevent microbial infection during and after the procedure. The subject is then placed in the dorsal lithotomy position, for example, and regional or general anesthesia is administered. Preparation of the subject may also include isolation of the anus 38 with a stapled towel or plastic drape (not shown). After appropriately positioning the subject, a Foley urethral catheter 39 (FIG. 4) is placed in the urethra 32.

Next, a peri-urethral site 40 (FIG. 3) at the anterior vaginal wall 24 of the subject is exposed so that the position of the urethra 32 can be determined. The mid-urethra is then injected with about 3 cc of normal saline to facilitate visualization of the urethra 32. After injecting the saline, a first stab incision 42 is made at the peri-urethral site 40. For example, the first stab incision 42 can be made in the upper vaginal wall beneath the bladder neck (not shown in detail) at the urethro-vesical junction (not shown in detail). Starting on one side of the urethra 32, the first stab incision 42 is made through the anterior vaginal wall 24 approximately 0.5 cm lateral and parallel to the midline of the urethra. A second stab incision 44 is next made through the anterior vaginal wall 24 approximately 0.5 cm lateral and parallel to the midline of the urethra 32.

After making the first and second stab incisions 42 and 44 through the anterior vaginal wall 24, third and fourth stab incisions 46 and 48 (FIG. 4) are made at the suprapubic region of the subject. As shown in FIG. 4, the third and fourth incisions 46 and 48 are made about 2 cm cephalad to the superior portion of the pubic bone 28. Next, a guide needle (not shown), such as a Stamey or Emmet needle is inserted into the third stab incision 46, guided through the abdominal tissue, and directed out of the first stab incision 42. The first and second end portions 16 and 18 of the urethral sling 12 are then attached to the guide needle. The guide needle is withdrawn through the third and fourth stab incisions 46 and 48 until the central support portion 14 of the urethral sling 12 is taught against the anterior vaginal wall 24 (FIGS. 5 and 6).

After the first and second end portions 16 and 18 of the urethral sling 12 are pulled through the third and fourth stab incisions 46 and 48, respectively, any excess of the first and second end portions are cut at the level of the subject's skin. The third and fourth stab incisions 46 and 48 are covered by adhesive bandages, and the first and second stab incisions 40 and 42 are covered by adhesive strips. With the central support portion 14 of the urethral sling 12 pulled taught against the anterior vaginal wall 24, pressure is exerted on the urethra 32 to lift the urethra back into a normal position and thereby aid in urine retention. The urethral sling 12 can be left in the subject or, after an appropriate period of time, removed from the subject in an outpatient setting.

From the above description of the invention, those skilled in the art will perceive improvements, changes and modifications. It will be appreciated that the method of the present invention is not intended to be limited to the order of steps described above. For example, the third and fourth stab incisions 46 and 48 can be made prior to making the first and second stab incisions 40 and 42. Additionally, it will be appreciated that the urethral sling 12 can include a radio-opaque material (not shown) to facilitate visualization during imaging-assisted implantation of the urethral sling. Such improvements, changes, and modifications are within the skill of the art and are intended to be covered by the appended claims.

Having described the invention, we claim:

1. An apparatus for treating urinary incontinence in a female subject, said apparatus comprising:
an elongated urethral sling having a central support portion intermediate first and second end portions, said central support portion including first and second ends respectively coupled to and extending from said first and second ends of said central support portion;
said central support portion being comprised of a non-resorbable polymer mesh;
said first and second end portions being comprised of a polymer mesh different than said non-resorbable polymer mesh.

2. The apparatus of claim 1, wherein said non-resorbable polymer mesh includes a synthetic polymer.

3. The apparatus of claim 2, wherein said synthetic polymer comprises polypropylene.

4. The apparatus of claim 1, wherein said polymer mesh includes a synthetic polymer.

5. The apparatus of claim 1, wherein the said first and second ends of said central support portion are in physical contact, but not integrally formed with, said first and second end portions, respectively.

6. The apparatus of claim 1, wherein said central support portion further includes a first major surface oppositely disposed from a second major surface.

7. The apparatus of claim 6, wherein either of said first and second major surfaces is adapted for placement against an anterior vaginal wall.

8. The apparatus of claim 7, wherein either of said first and second major surfaces has a shape that substantially mirrors the shape of the anterior vaginal wall.

9. A method for treating urinary incontinence in a female subject, said method comprising the steps of:
providing an elongated urethral sling having a central support portion intermediate first and second end portions, the central support portion including first and second ends, the first and second end portions being respectively coupled to and extending from the first and second ends of the central support portion, the central support portion being comprised of a non-resorbable polymer mesh and the first and second end portions being comprised of a polymer mesh different than the non-resorbable polymer mesh; and
implanting the urethral sling in the subject so that the central support portion is taught against the anterior vaginal wall and provides support to the urethra.

10. The method of claim 9, wherein said step of implanting the urethral sling in the subject further comprises the steps of:
making first and second incisions at a peri-urethral site, the first and second incisions being substantially parallel to one another and lateral to the midline of the urethra on the anterior vaginal wall; and
making third and fourth incisions at a suprapubic region of the subject.

11. The method of claim 10 further comprising the steps of:
inserting a delivery needle into the third incision;
advancing the delivery needle through the third incision and out of the first incision;
operably coupling the first and second end portions of the urethral sling to the delivery needle; and
manipulating the delivery needle so that the first and second end portions of the urethral sling exit the third and fourth incisions.

12. The method of claim 11 further comprising the step of manipulating the first and second end portions of the urethral sling to pull the central support portion taught against the anterior vaginal wall.
